(19) United States
(12) Patent Application Publication
(10) Pub. No.: US 2023/0255547 A1
(43) Pub. Date: Aug. 17, 2023

(54) METHOD FOR DETERMINING A SCALP TYPE OF A USER

(71) Applicant: Henkel AG & Co. KGaA, Duesseldorf (DE)

(72) Inventors: Yi SHI, Shanghai (CN); Xiaowei CHANG, Shanghai (CN)

(73) Assignee: Henkel AG & Co. KGaA, Duesseldorf (DE)

(21) Appl. No.: 18/003,242

(22) PCT Filed: Jun. 30, 2020
(86) PCT No.: PCT/CN2020/099150

§ 371 Date: Dec. 23, 2022

(51) Int. Cl. A61B 5/00; A61B 5/0537; G16H 20/10

(52) U.S. Cl. CPC A61B 5/446; A61B 5/443; A61B 5/0537; A61B 5/0077; A61B 5/0071; A61B 5/7267; G16H 20/10; A61B 2576/02

(57) ABSTRACT

A method determining a scalp type of a user is provided. A value indicative of a humidity level of a region of the scalp of the user and a value indicative of a sebum level in the region of the scalp of the user are obtained. The set comprising both values is compared to reference groups of scalp hydration and sebum levels, the reference groups forming at least five scalp types. Each scalp type is associated with a humidity level range and a sebum level range, and with a corresponding category of skin and/or hair care compositions and/or skin and/or hair care treatments compatible with said humidity level range and sebum level range. Based on the comparison, the scalp type of the user is identified and then outputted.

### CROSS-REFERENCE TO RELATED APPLICATION

This application is a U.S. National-Stage entry under 35 U.S.C. § 371 based on International Application No. PCT/CN2020/099150, filed Jun. 30, 2020, which was published under PCT Article 21(2), which is all hereby incorporated in it's entirety by reference.

### TECHNICAL FIELD

The present disclosure relates to the field of skin and hair care, and more particularly to methods for determining the scalp type of a user in order to classify the scalp type into a group associated with suitable skin and hair care product or treatment recommendations. The present disclosure more particularly aims to render the determination of the scalp type more objective to avoid a false assessment of the scalp condition which can lead to the selection of unsuitable scalp or hair care products, and more convenient for a user.

### BACKGROUND

Hair care involves the selection of products or treatments compatible with the state of a person's hair. Damaged hair may typically require products that will preserve the structure of hair without inducing further damage. Hair care is not independent from skin care in particular scalp care since the scalp condition may have a strong impact on the general appearance of a person's hairstyle and contribute either to a healthy and appealing look or provide a negative general impression, for example in the event of redness, oiliness, or presence of dandruff.

Despite the relevance of selecting treatments and products that preserve both hair and scalp, there is no general rule for the selection of such products or treatments. Some people typically suffer from a higher sebum secretion on their scalp and therefore would benefit from treatments and products that reduce the level of sebum on their scalp, whereas for other people the opposite occurs.

It is however difficult to make an accurate assessment of one's scalp condition, as this part of the skin generally requires a third party's intervention to be examined and is most of the time hidden under hair.

A visual assessment of a person's scalp condition is generally difficult and results in inaccurate observations. Oiliness and moisture can lead to similar visual assessments although they would not trigger the same treatment recommendation.

In order to assess the scalp type of a user with more accuracy, it is possible to use biophysical measurements of the skin conditions. Typically, electrodes can be used to infer moisture level from the measured conductivity of the skin. Oiliness can be determined as a sebum level using translucent tapes that contact the scalp thereby absorbing sebum. The presence of sebum on the tapes changes their translucency which can be measured optically. Such measurements only provide a set of raw data that can help assess the true condition of the scalp of a user. However, individual measurements are hard to interpret without any comparison means to other scalps.

Document US 20190237194 A1 describes one example of a method for assessing the scalp of a user and recommend a skin treatment product. The assessment of the scalp type relies on measurements of hydration and sebum compiled together with further information provided by the user or by other complementary measurements. All the provided information is converted into a health score to determine a type of scalp treatment.

This approach requires several inputs from a user in order to accurately assess his scalp condition. Such an approach is not convenient for users who do not have much time to spend answering questionnaires or who simply do not know how to answer the questions and add an element of subjectivity to the assessment of the health score.

Furthermore, assessing one's scalp type is a continuous process as scalp condition changes after each hair washing step and this may lead to a change in the recommendable hair and/or scalp treatment products or treatments.

A method for simply and objectively assessing the scalp type of user is therefore sought.

### BRIEF SUMMARY

To address the above need, the present disclosure provides a method for determining a scalp type of a user, the method comprising:

obtaining a value indicative of a humidity level of a region of the scalp of the user;

obtaining a value indicative of a sebum level in the region of the scalp of the user;

comparing a set comprising the obtained value indicative of the humidity level and the value indicative of the sebum level to reference ranges of scalp hydration and sebum levels, the reference ranges forming at least five scalp groups, each scalp group being associated with a humidity level range and a sebum level range, each scalp group being further associated with a corresponding category of skin and/or hair care compositions and/or skin and/or hair care treatments compatible with said humidity level range and sebum level range;

identifying, based on the comparison, a scalp type of the user among the at least five scalp groups; and

outputting the identified scalp type.

The present disclosure overcomes the burden of subjectively assessing the scalp type of a user through a visual observation by providing a simple and objectivized approach to determining the scalp type of a user that can be implemented with a portable device and a processor or computer which can be part of the portable device. To enable such an automatic and objectivized assessment of the scalp condition of a user, only two related parameters need to be measured: the humidity level and the sebum level, obtained for a same region of a scalp of a user. Applicant noticed that scalps typically come in five distinct types. These types typically are associated with the type of product or treatment that would best suit the scalp type. Although morphological features within each type might be identical, Applicant noticed that it is possible to exhaustively describe all five scalp types with particular ranges for the humidity and sebum level. The value ranges for humidity and sebum were acquired on a representative batch of users whose scalp type was further measured with optical and biophysical measurement tools. The obtained sebum values and humidity values were plotted in two dimensions and thresholds for each range of sebum and humidity level was found to match five scalp types. Each scalp type is exemplified by a particular category of products or treatments recommendable to treat hair or scalp.

The method of the present disclosure consists in comparing measured values for sebum level and humidity level of the scalp of a user to the ranges that were defined on the representative batch of users. This allows a user to know the nature of his scalp condition and choose an appropriate hair or scalp care product and/or hair or scalp treatment.

According to an embodiment, the at least five scalp groups may be established on the basis of data extracted from scalps of at least one hundred users using at least one among:

measurements of biophysical parameter values of the scalps, the biophysical parameter values comprising a hydration level value and a sebum level value for each scalp; and

an analysis of scalp features on magnified photographs of the scalps of the at least one hundred users.

Optionally, the biophysical parameter values and/or the scalp features may be grouped into five clusters based on similarity of biophysical parameter values and/or scalp features.

According to an embodiment, the value indicative of the humidity level of the region of the scalp of the user and the value indicative of the sebum level of the region of the scalp of the user may be obtained using at least one among:

measurements of biophysical parameter values of the region of the scalp of the user, the biophysical parameter values comprising a hydration level value and a sebum level value of the region of the scalp of the user; and

an analysis of scalp features on magnified photographs of the region of the scalp of the user.

According to an embodiment, the measurement of biophysical parameter values of the scalps may comprise electrical conductivity measurements or electrical resistance measurements on an upper layer of the scalp to obtain values of hydration levels and measurements of changes of translucency of absorbing strips put in contact with the scalps to obtain sebum levels.

It is to be noted that “electrical conductivity measurements” are equivalent within the meaning of this present disclosure to “electrical resistance measurements”. It is to be noted that any other alternative method for measuring the sebum level or humidity level can be used, for example relying on infrared cameras or dedicated measurement devices.

According to an embodiment, the features on the magnified photographs may comprise at least one among:

an intensity of light reflected by the region of the scalp;

a count of dandruff on the magnified photograph;

a count of red portions on the magnified photograph, the red portions being indicative of inflammations on the scalp; and

an amount of fluorescent light emitted from follicles of the scalp,

wherein each scalp is photographed under natural light, ultraviolet light and polarized light conditions.

Typically such features may be counted per unit area on the images, and the images may either be taken in specific conditions of illumination intensity, viewing angle or distance to the scalp, or be later calibrated to compensate for any differences with respect to reference conditions. Such counts can be implemented on the reference batch of scalps used to set the ranges of humidity and sebum values for all the five scalp groups and also later on each scalp to be assessed.

According to an embodiment, the method may further comprise:

defining the at least five scalp groups based on a visual assessment of dryness and oiliness of the scalps of the at least one hundred users, the visual assessment corresponding, for each scalp group of the at least five scalp groups, to an assessment of a similarity in the skin and/or hair care compositions and/or skin and/or hair care treatments adapted for said scalp group.

This optional approach to defining the five scalp groups relies on a professional's assessment of the type of product or treatment that would be recommended for a scalp of a user. The assessment is performed on a representative batch of users and their scalps are grouped according to similarities in the recommendations provided by the professional. Such an approach can also be used in conjunction with the biophysical measurements approach and/or the image analysis approach in order to adjust the threshold of values for each scalp group.

According to an embodiment, the reference ranges may be established on the basis of data extracted from scalps of at least one hundred users, the method further comprising, for each of the at least one hundred users:

obtaining magnified photographs of the scalps of the at least one hundred users;

grouping the obtained magnified photographs according to the defined at least five scalp groups; and

for each scalp group of the at least five scalp groups, determining combinations of features found within grouped magnified photographs of scalps belonging to said scalp group.

Such an approach consists in a learning process during which features on images are identified based on scalps that have already been classified into each of the least five scalp groups. The process can for example be implemented by a human eye counting features on the magnified photographs to determine the combination of features of the scalps that can be used to match each scalp group that was already defined with ranges of humidity levels and sebum levels.

According to an embodiment, the at least five scalp groups may comprise:

a first scalp group corresponding to a hydration level measurable as an upper skin conductivity of 0 μS to 40 μS and a Sebum level measurable as a reduction in translucency of a sebum-absorbing tape film that had been in contact with the scalp of the user between 0% and 20%;

a second scalp group corresponding to a hydration level measurable as an upper skin conductivity of 0 μS to 80 μS and a Sebum level measurable as a reduction in translucency of a sebum-absorbing tape film that had been in contact with the scalp of the user between 20% and 60%;

a third scalp group corresponding to a hydration level measurable as an upper skin conductivity of 0 μS to 80 μS and a Sebum level measurable as a reduction in translucency of a sebum-absorbing tape film that had been in contact with the scalp of the user between 60% and 100%;

a fourth scalp group corresponding to a hydration level measurable as an upper skin conductivity of 40 μS to 80 μS and a Sebum level measurable as a reduction in translucency of a sebum-absorbing tape film that had been in contact with the scalp of the user between 0% and 40%; and

a fifth scalp group corresponding to a hydration level measurable as an upper skin conductivity at or above 80 μS and a Sebum level measurable as a reduction in translucency of a sebum-absorbing tape film that had been in contact with the scalp of the user between 0% and 60%.

According to embodiment, the scalp groups may further comprise the following features:

the first scalp group is associated with a corresponding category of skin and/or hair care compositions comprising between 10% and 18% surfactants, and have a Zein value lower than 0.75;

the second scalp group is associated with a corresponding category of skin and/or hair care compositions comprising between 12% and 20% surfactants, and have a Zein value lower than 1;

the third scalp group is associated with a corresponding category of skin and/or hair care compositions comprising between 14% and 20% surfactants, and have a Zein value lower than 2.5;

the fourth scalp group is associated with a corresponding category of skin and/or hair care compositions comprising between 10% and 18% surfactants, and have a Zein value lower than 2; and

the fifth scalp group is associated with a corresponding category of skin and/or hair care compositions comprising between 10% and 18% surfactants, and have a Zein value lower than 1.5.

According to an embodiment, the scalp groups may further comprise the following features:

the first scalp group is associated with a corresponding category of skin and/or hair care compositions comprising between 0% and 2% of an anti-dandruff agent;

the second scalp group is associated with a corresponding category of skin and/or hair care compositions comprising between 0% and 2% of an anti-dandruff agent;

the third scalp group is associated with a corresponding category of skin and/or hair care compositions comprising between 0% and 1% of an anti-dandruff agent;

the fourth scalp group is associated with a corresponding category of skin and/or hair care compositions void of anti-dandruff agent; and

the fifth scalp group is associated with a corresponding category of skin and/or hair care compositions void of anti-dandruff agent.

The method of the present disclosure further pertains to a method for recommending a skin or hair care product and/or a skin or hair care treatment to a user comprising:

determining a scalp type of the user with a method as described above;

determining, based on the identified scalp type of the user, the corresponding category of skin and/or hair care compositions and/or skin and/or hair care treatments compatible with the humidity level range and sebum level range of the scalp type of the user; and

outputting the corresponding category of skin and/or hair care compositions and/or skin and/or hair care treatments.

This method may advantageously be implemented with a processor, part of a portable device or a computer. Information regarding the category of skin and/or hair care compositions and/or skin and/or hair care treatments compatible with the humidity level range and sebum level range of the scalp type of the user may be stored on a local database or accessed at a distant computer/server or in a cloud environment. The database may also be updated using either user feedback or to take into account evolutions and improvements in the matching method described above.

According to an embodiment, the method for recommending a skin or hair care product and/or a skin or hair care treatment to a user may further comprise:

obtaining a magnified photograph of the region of the scalp of the user; and

upon identifying a number of red portions on the magnified photograph that is above a predetermined threshold, recommend a skin and/or hair care composition comprising a reduced proportion of surfactants adapted for sensitive scalps.

According to an embodiment, the method for recommending a skin or hair care product and/or a skin or hair care treatment to a user may further comprise:

comparing a current value indicative of the humidity level of the region of the scalp of the user and a current value indicative of the sebum level of a region of the scalp of the user to previously obtained values for the region of the scalp of the user;

upon determining that the sebum level of the region of the scalp of the user increases, output a recommendation to increase the frequency of hair wash using a hair care product compatible with the scalp type of the user; and

upon determining that the sebum level of the region of the scalp of the user decreases, output a recommendation to use a hair or scalp care product comprising a lower proportion of surfactants.

In such a dynamic process of determining the evolution over time of the scalp of the user suitable products or treatment recommendations may differ from those normally recommended for a given scalp type, in order to stimulate a desired change of the scalp condition into a condition preset by the user or recommended by a hair care professional for example.

The present disclosure also pertains to a computer program product comprising instructions for executing a method for determining a scalp type of a user, the method comprising:

obtaining, as an input, a value indicative of a humidity level of a region of the scalp of the user;

obtaining, as an input, a value indicative of a sebum level in the region of the scalp of the user;

comparing a set comprising the obtained value indicative of the humidity level and the value indicative of the sebum level to reference ranges of scalp hydration and sebum levels, the reference ranges forming at least five scalp groups, each scalp group being associated with a humidity level range and a sebum level range, each scalp group being further associated with a corresponding category of skin and/or hair care compositions and/or skin and/or hair care treatments compatible with said humidity level range and sebum level range;

identifying, based on the comparison, a scalp type of the user among the at least five scalp groups; and

outputting the identified scalp type.

In other words, the present disclosure concerns a non-transitory computer readable storage medium having stored thereon a computer program comprising instructions for execution of the method described above.

### BRIEF DESCRIPTION OF THE DRAWINGS

The present disclosure will hereinafter be described in conjunction with the following drawing figures, wherein like numerals denote like elements, and:

FIG. 1 shows a simplified workflow of a possible method according to an exemplary embodiment;

FIG. 2 is a schematic representation of methods for measuring physiological parameters of the scalp of a user according to an embodiment;

FIG. 4 is a diagram showing a distribution of points corresponding to humidity levels (horizontal axis) and sebum levels (vertical axis) measured on a batch of users, showing a clustering of the points into five scalp groups;

FIG. 4a is a schematic representation of method for acquiring images of the scalp of a user for features analysis thereof; and

FIG. 4b is a schematic representation of a picture of the scalp of a user showing feature that can be found thereon.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present disclosure provides a method for determining the scalp type of a user using a minimal amount of parameters from the scalp of the user and based on an objective assessment of the scalp condition.

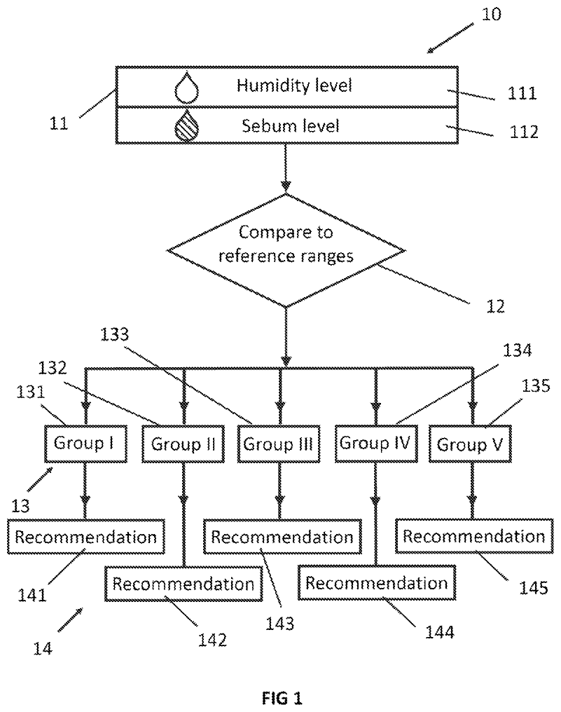

As seen on FIG. 1, the method 10 for determining a scalp type of a user starts with a step 11 by obtaining a hydration level 111 and a sebum level 112 on a region of a scalp of a user. There is no limitation to the size or location of the region of the scalp of a user, however it is preferable to choose a section of the scalp where hair density does not prevent measuring devices from accessing the skin on the head of the user.

Regardless of the unit in which the sebum level and the hydration level are expressed, these may advantageously be converted into a conductivity value equivalent for the hydration level value, for example per unit area of the scalp surface, and a percentage of light transmissivity for the sebum level value. The light transmissivity value describes the percentage of reduction in light that can pass through a standard strip of paper put into contact with the scalp of the user to soak sebum present thereon. Other units can be used to describe the ranges and features of each scalp group.

Values for these two parameters that describe the greasiness of the scalp (via the sebum level value) and the moisture of the scalp (via the humidity level value) are compared at step 12 to reference ranges for both parameters.

These reference ranges define at least five scalp groups 131-135, which are defined based on the type of treatments or products (in particular categories of skin and/or hair care compositions and/or skin and/or hair care treatments 141-145) that would typically be suitable or recommendable to be used with each scalp group 131-135. Applicant noticed that sebum and humidity are two particularly suitable parameters to assess a scalp type. This observation comes from a thorough study of more than one hundred representative users of different ages and genders.

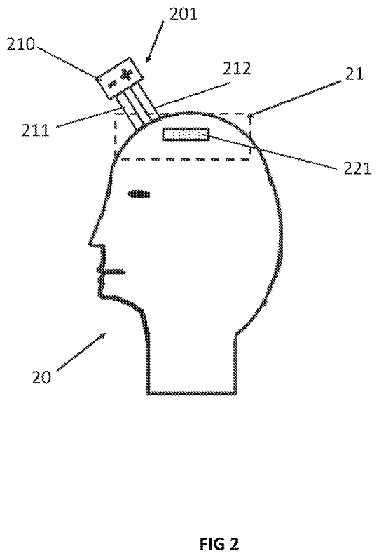

On this representative batch of users, hydration levels of the scalp were measured using a measuring device typically comprising electrodes as represented on FIG. 2. Such a device 201 comprises at least two electrodes 211, 212 and a controller 210 for measuring a current that passes through the electrodes and region of the scalp 21 of the user 20. Such a device gives access to the hydration levels of the stratum corneum layer of the scalp and outputs a value in Siemens (unit S or μS) representative of the scalp conductivity per unit area of scalp. As further seen on FIG. 2, a measurement of sebum levels can be obtained by applying a standard strip 221 of paper on the region of the scalp 21 of the user 20. By absorbing sebum present on the scalp, the strip 221 of paper will see a change in its translucency. 0% indicates no change in translucency (indicative of the lowest possible sebum concentration on the scalp of a user, so that the translucency of the standard strip of paper does not change at all with respect to a strip that was not in contact with the scalp) and 100% indicates a full decrease in translucency compared to a strip with no sebum thereon (indicative of a highest measurable sebum level on the scalp of the user).

Other standards for defining and measuring the scalp humidity level and scalp sebum level can be used.

Further instruments can also be used to measure trans-epidermal water loss on the scalp, pH values of the scalp, in order to obtain a fuller picture of the condition of the scalp of the users 20 from the representative batch of users.

Once these parameters are measured for all users 20 of the representative batch of users, the values of sebum level versus humidity level are plotted in a two-dimensional diagram as represented on FIG. 3.

Vertical axis 302 on FIG. 3 shows sebum level values in arbitrary units (corresponding to the translucency measured with a standard strip of paper as explained above). These units can be converted into any other unit depending on the type of method used for determining the sebum level.

Horizontal axis on FIG. 3 shows the hydration level expressed as a conductivity of the stratum corneum measured in microSiemens (μS). These units can be converted into any other unit depending on the type of method used for determining the hydration level.

As can be seen on FIG. 3, the distribution of data points is not homogenous across all the two-dimensional area of the graph. Most users have moderately low sebum and hydration levels of their scalps, which would generally be considered as a “normal” scalp condition, or a scalp condition that would naturally occur shortly after washing one's hair.

Each individual scalp condition represented on FIG. 3 is also separately assessed with a hair care professional to determine the most suitable range of categories of products for hair or scalp treatment. From this assessment and from using a clustering algorithm, it has been found that scalp types can be divided into the at least five groups shown on FIG. 3.

A first scalp group 1 typically corresponds to hydration levels measurable as an upper skin conductivity of 0 μS to 40 μS and sebum levels measurable as a reduction in translucency of a sebum-absorbing tape film that had been in contact with the scalp of the user between 0% and 20%. This group in FIG. 3 encompasses about 47% of all user scalp types. This group typically corresponds to scalps that are not very moist and not very greasy.

A second scalp group 2 typically corresponds to hydration levels measurable as an upper skin conductivity of 0 μS to 80 μS and sebum levels measurable as a reduction in translucency of a sebum-absorbing tape film that had been in contact with the scalp of the user between 20% and 60%. One notices on FIG. 3 that some points from the second scalp group 2 seem to partially overlap with points that would typically be graded by hair care professionals as being suitable for other categories of scalp or hair care products and/or scalp and/or hair care treatments, namely those of scalp group 4. This is not a matter for concern as the boundaries between adjacent scalp group types would not necessarily lead to incompatible recommendations. The scalps of the second scalp group 2 typically correspond to scalps that are not very moist and slightly greasy. A total of about 15% of all scalps observed in the representative batch fall into this scalp group. One frequent evolution that can for example be observed when a user does not wash his hair for some time is a tendency to move from the first scalp group to the second scalp group. Therefore, based on the frequency on which a person washes his hair, different types of hair or scalp products might be suitable for ensuring the scalp stays healthy. It is further to be noted that a healthy scalp typically has moisture and greasiness values that fit the values observed for the first scalp group 1.

A third scalp group 3 typically corresponds to hydration levels measurable as an upper skin conductivity of 0 μS to 80 μS and sebum levels measurable as a reduction in translucency of a sebum-absorbing tape film that had been in contact with the scalp of the user between 60% and 100%. As seen on FIG. 3, the actual boundary in terms of measured hydration levels can be extended to conductivities above 80 μS, although frequency of occurrence of such extreme cases is very low. Scalps of the third scalp group 3 typically corresponds the greasiest scalps observed and encompass about 8% of all scalps observed in the experiments conducted by the Applicant. This would be considered as not esthetical to the naked eye and would therefore be associated with hair or scalp care products that significantly reduce oiliness of skin. Such products would generally be considered as too aggressive and potentially slightly damageable to the hair in other scalp types but would not affect the scalp or hair of users from the third scalp group 3.

A fourth scalp group 4 typically corresponds to hydration levels measurable as an upper skin conductivity of 40 μS to 80 μS and sebum levels measurable as a reduction in translucency of a sebum-absorbing tape film that had been in contact with the scalp of the user between 0% and 40%. Some of the points on the diagram of FIG. 3 belong simultaneously to the type of the second scalp group 2 and the type of the fourth scalp group, especially for sebum levels between 20% and 40% and hydration levels expressed as a conductance between 40 μS and 80 μS. As explained above, this does not have a significant impact on the product recommendation as at these intermediate values the wish to clean hair in order to reduce greasiness is the general recommendation in terms of scalp and/or hair care treatments or products. No further action on hydration is deemed to be required as hydration is not esthetically perceived as being a sign of unhealthy or dirty hair or scalp. About 22% of all users had a scalp type belonging to the fourth scalp group 4.

Finally, FIG. 3 shows a fifth scalp group 5 corresponding typically to hydration levels measurable as an upper skin conductivity at or above 80 μS and sebum levels measurable as a reduction in translucency of a sebum-absorbing tape film that had been in contact with the scalp of the user between 0% and 60%. This group encompasses about 8% of all scalp groups observed in the experiment conducted by the Applicant.

It could be summarized that users whose scalp is identified as belonging to the third scalp group 3 would wish to reduce the greasiness of their hair. It could be that this greasiness is only a temporary result of a longer than usual period spent without washing one's hair or a temporary change in diet. Users whose scalp type is determined as belonging to the fifth scalp group 5 may wish to reduce the moist level of their scalp. A sweaty scalp is typically considered as more likely to attract residues from grooming or polluting particles. It is generally admitted that products with more surfactant content are best suited for such scalp types (with 10% to 18% surfactants for example).

Another aspect of the present disclosure concerns a time-dependent monitoring of the evolution of the scalp type of one user. A user whose scalp type is identified as on average belonging to one of the first scalp group 1, second scalp group 2 or fourth scalp group 4 most of the time, would be surprised to notice a sudden change in his scalp group if it switches to the third or fifth scalp groups. This could trigger further awareness that an unusual change in either diet, daily routine or a particular health condition is affecting the scalp of the user. One recommendation, beyond hair or scalp treatments or application of specific hair or scalp care products could also be seeking advice from a professional or provision of specific advice on diet or routine fitness activities that could affect moisture or greasiness of scalps.

A user's scalp type can therefore be easily determined by comparing a set of values comprising a hydration level value and a sebum level value of the region of the scalp 21 of the user 20 to references ranges as those shown on the diagram of FIG. 3.

It is to be noted that, besides the measurement of these two biophysical parameters to determine the scalp type of a user, it is also possible to analyze the content of images of the scalp of a user.

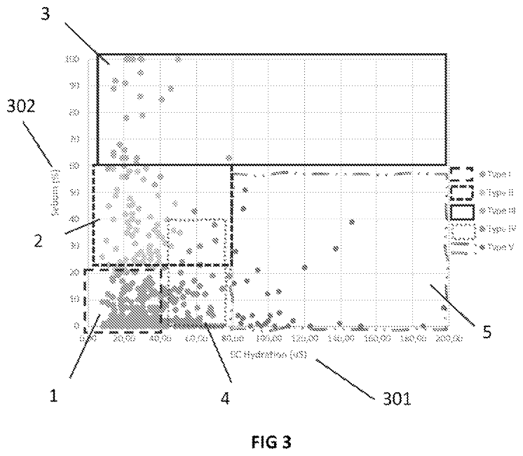

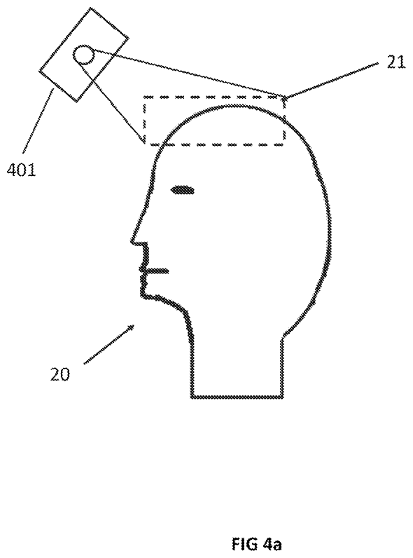

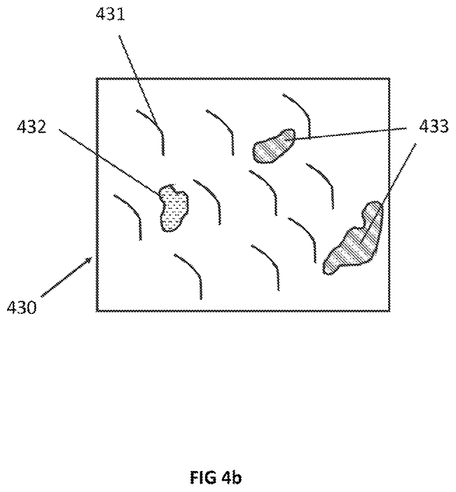

FIG. 4a is a schematic drawing of an image acquisition device 401 capturing light reflected from the region of the scalp 21 of a user 20. Although not represented, the acquisition device 401 may typically comprise a light emitting unit that is capable of casting ultraviolet light (with wavelengths ranging from 100 nm to 400 nm) or polarized light (linearly polarized light in particular) onto the region of the scalp 21 of the user 20. A source of visible light (with wavelengths typically ranging from 400 nm to 800 nm) may also be present. A corresponding sensor unit is also included in the image acquisition device 401. Image acquisition device 401 also advantageous takes magnified images of the region of the scalp 21 of the user 20, for example with a magnification factor of about 80.

An example of a magnified image 430 of the region of the scalp 21 of the user 20 is shown on FIG. 4b. This magnified image 430 comprises hairs 431, red portions 432 and signs of dandruff 433.

Typically, image analysis consists in looking at the intensity of visible light reflected from the scalp on the image to determine the humidity level of the scalp. A flaky state of hair, with the presence of dandruff 433 typically is a sign of low moisture of the scalp. Based on the number of dandruff 433 portions (their size and number over the magnified image 430) it is possible to qualitatively grade the humidity level as high, slight or low.

Polarized images emphasize micro-inflammation or higher sensitivity areas of the scalp. Erythema, appearance of blood capillaries typically seen as red portions 432 are signs of a higher sensitivity. Based on the amount of these areas (their respective size over the whole magnified image 430) a qualitative score such as high, slight, or low can be given to the scalp. Under ultra-violet (UV) light, follicular clogging can be detected. Orange or red fluorescence areas in the vicinity of hair roots can be seen on the magnified images (not shown). This fluorescence is typically due to porphyrin fluorescence under UV light illumination. Porphyrins are produced by lipid-fed bacteria called Cutibacterium acnes, which therefore gives insights into the sebum level of the scalp. Follicular clogging (and sebum level) can qualitatively be graded as high, slight or low from the magnified image 430.

Table 1 shown below summarizes the features that can typically be found in each scalp groups defined above using either the biophysical parameters defined above or the image analysis description.

TABLE 1
correspondence between reference ranges from biophysical
measurement of humidity level and sebum level and
features count on images across all five scalp groups

| | Biophysical parameter- | Imaging based |
|---|---|---|
| Scalp groups | based Classification | Classification |
| Group 1 | Low hydration level | slight to high |
| Low greasiness | (0-40 μS) | dandruff level |
| Low moisture | Low sebum level (0-20%) | Low reflectivity |
| Group 2 | Low hydration level (0-80 μS), | Slight fluorescence |
| Low moisture | Moderately high sebum | no-to-slight |
| Moderately high | level (20%-60%) | dandruff level |
| greasiness | | |
| Group 3 | Low hydration level | high fluorescence, |
| Low moisture | (0-80 μS, >80 μS), | no-slight dandruff |
| Extremely high | Extremely high sebum | |
| greasiness | level (>60%) | |
| Group 4 | Moderately high hydration | Moderately high |
| Moderately high | level (40 μS-80 μS), | reflectivity, |
| moisture | Low sebum level (0-40%) | Low-medium |
| | | fluorescence |
| Low greasiness | | No-slight dandruff |
| Group 5 | Extreme high hydration | High reflectivity, |
| Extremely high | level (>80 μS), | no dandruff |
| moisture | Low sebum level (0-60%) | |
| Low greasiness | | |

Now that a method for determining the scalp type of a user has been described, a further method using the determined scalp type to recommend a skin or hair care product and/or a skin or hair care treatment to a user is described.

As explained above, each scalp group is defined based on the category of skin and/or hair care compositions and/or skin and/or hair care treatments compatible with the humidity level range and sebum level observed in that group.

Typically, for users that have a scalp type belonging to the first scalp group 1, it is sought to increase scalp hydration to avoid abnormal desquamation and in order to reduce scalp sensitivity.

To achieve this, surfactants used in shampoo formulas for this scalp type should be mild, avoid irritation to the scalp barrier. The total level of surfactants should not exceed a certain threshold to avoid further excessive reduction of sebum from the scalp. Anti-dandruff agents may be used to cure flakes and maintain a healthy scalp environment. Typical ranges would therefore comprise 10% to 18% surfactants, a total amount of anti-dandruff agent between 0% and 2% and a Zein value inferior to 0.75. The Zein value defines the number of grams of zein that can be dissolved by every gram of surfactant. Zein is a corn protein that is considered to be similar to keratin present in the skin and hair. Zein value is an indicator of the irritation potential of a surfactant or surfactant-based product.

Typically, for users that have a scalp type belonging to the second scalp group 2, it is sought to improve the water-oil imbalance on the scalp, by increasing the moisture level and or reducing the sebum level, so that the redness, sensitivity of the scalp decreases.

To achieve this, surfactants used in shampoo formulas for this scalp type should offer a balance between mildness for the scalp and sufficient cleaning efficiency of the shampoo. Anti-dandruff agents may be used to cure flakes and maintain a healthy scalp environment. Typical ranges would therefore comprise 12% to 20% surfactants, a total amount of anti-dandruff agent between 0% and 2% and a Zein value inferior to 1.

Typically, for users that have a scalp type belonging to the third scalp group 3, it is sought to reduce the sebum concentration on the scalp of the user. Sufficient cleaning with more surfactants is therefore recommended to avoid severe follicular clogging, unhealthy follicles irritations and greasy, sticky hair.

To achieve this, the total level of surfactants be high enough. Some anti-dandruff agents may also be present as long as they do not reduce the cleaning efficiency conferred by the surfactants. Typical ranges would therefore comprise 14% to 20% surfactants, a total amount of anti-dandruff agent between 0% and 1% and a Zein value inferior to 2.5.

It is generally recommended to reassess the scalp type of a user within 24 hours after washing one's hair to avoid any significant influence of the accumulated dirtiness of the hair and scalp on the assessment of the scalp type of the user.

Typically, for users that have a scalp type belonging to the fourth scalp group 4, no special effect is sought other than maintaining values within the ranges of this scalp group. Scalps of this scalp group are generally strongly resilient to external influences (chemical treatments of hair, hair coloration, weather conditions for example).

As a result, there is no special restriction as to the type of surfactants or their total level to be used, and anti-dandruff agents re generally not required. Typical ranges would therefore comprise 10% to 18% surfactants and a Zein value inferior to 2.

Typically, for users that have a scalp type belonging to the fifth scalp group 5, it is sought to achieve a better cleaning of the scalp to remove any pollution particles or residues from grooming that accumulate on sweaty scalps, and avoid proliferation of micro-organisms on the scalp that can lead to irritations that render the scalp more sensitive.

To achieve this, some surfactants should be present to ensure proper cleansing of the scalp but they should be moderately mild. Anti-dandruff agents are optional. Typical ranges would therefore comprise 10% to 18% surfactants and a Zein value inferior to 1.5.

The above categories of product or treatment recommendations can be further enhanced by adding information from an analysis of features on a magnified image of the scalp of the user.

Indeed, a thorough count of these features can allow a finer tuning of each of the above recommendations to make an even more personalized scalp and hair care assessment of the scalp of the user.

For example, in the event of high sensitivity shown as a high number of red areas on the scalp, it is recommended to put a stronger focus on the mildness of the surfactants used in the shampoo.

Anti dandruff agents can be increased or decreased in concentration in the final shampoo formula based on the amount of dandruff seen on the magnified images.

As for follicular clogging (seen as a higher or lower presence of fluorescent traces on images under UV light illumination, which reflects a higher or lower sebum concentration), they are not only representative of sebum concentration but are an indicator of the interaction between hair and scalp at the hair roots. In case of a high number of follicular clogging, it would be recommended to increase the frequency of hair washing, and to adjust the surfactant concentration (by increasing its concentration for higher follicular clogging levels), to reduce the number of follicular clogging to better preserve hair.

Surfactant used in shampoo can typically be selected among anionic surfactants, non-anionic surfactants, amphoteric surfactants.

Anionic surfactants can typically comprise at least one among: Acylated Amino Acid such as for example Sodium Lauroyl Sarcosinate or TEA-Cocoyl Glutamate; Carboxylic Acids and salts such as for example Sodium Laureth-13 Carboxylate; sulfonic Acid derivatives such as for example Sodium Methyl Cocoyl Taurate, Sodium Cocoyl Isethionate or Sodium C14-16 Olefin Sulfonate; Sulfuric Acid derivatives such as for example Sodium Laureth Sulfate or Ammonium Lauryl Sulfate.

Non-anionic surfactants can typically comprise at least one among: Alkyl Glucosides: such as for example Coco-Glucoside or Lauryl Glucoside; Amine oxides such as for example Lauramine Oxide.

Amphoteric surfactants can typically comprise at least one among: Alkyl amido Alkyl Amines such as for example Sodium Lauroamphoacetate; Alkyl Betaines such as for example Cocamidopropyl betaine or Coco-betaine, coco-hydroxysultaine.

Typical anti-dandruff (AD) agents that could be found in shampoo include one among: Zinc pyrithione, Piroctone Olamine, Ketoconazole, Climbazole, Hexamidine diisethionate or Apium graveolens (Celery) Seed Extract for example.

The following table 2 summarizes the different recommendations described above.

TABLE 2
type of typically shampoo compositions suitable
for each scalp group further based on
features extracted from an image analysis of the scalp.

| | Sensitivity: | | Sensitivity: | Sensitivity: |
|---|---|---|---|---|
| | “high” | Sensitivity: | “Slight”/“no” | “Slight”/ |
| 5 different | Dandruff: | “high” | Dandruff: | “no” |
| Scalp | “high”/ | Dandruff: | “high”/ | Dandruff: |
| Conditions | “slight” | “No” | “slight” | “No” |
| Group 1 | Surfactants: | Surfactants: | Surfactants: | Surfactants: |
| Low moisture, | 10%~18% | 10%~18% | 10%~18% | 10%~18% |
| Low | Zein: <0.5 | Zein: <0.5 | Zein: <0.75 | Zein: <0.75 |
| greasiness | AD: | AD: | AD: | AD: |
| | 0.2%~2% | 0%~0.5% | 0.2%~2% | 0%~0.5% |
| Group 2 | Surfactants: | Surfactants: | Surfactants: | Surfactants: |
| Low moisture, | 12%~20% | 12%~20% | 12%~20% | 12%~20% |
| Moderately | Zein: <0.5 | Zein: <0.5 | Zein: <1 | Zein: <1 |
| high | AD: | AD: | AD | AD: |
| greasiness | 0.2%~2% | 0%~0.5% | 0.2%~2% | 0%~0.5% |
| Group 3 | Surfactants: | Surfactants: | Surfactants: | Surfactants: |
| Low moisture, | 14%~20% | 14%~20% | 14%~20% | 14%~20% |
| Extremely | Zein: <1 | Zein: <1 | Zein: <2.5 | Zein: <2.5 |
| high | AD: | AD: | AD: | AD: |
| greasiness | 0.2%~2% | 0%~0.5% | 0.2%~2% | 0%~0.5% |
| Group 4 | Surfactants: | Surfactants: | Surfactants: | Surfactants: |
| Moderately | 10%~18% | 10%~18% | 10%~18% | 10%~18% |
| high moisture, | Zein: <1 | Zein: <1 | Zein: <2 | Zein: <2 |
| Low | AD: | AD: / | AD: | AD: / |
| greasiness | 0.2%~2% | | 0.2%~2% | |
| Group 5 | Surfactants: | Surfactants: | Surfactants: | Surfactants: |
| Extremely high | 10%~18% | 10%~18% | 10%~18% | 10%~18% |
| moisture, | Zein: <1 | Zein: <1 | Zein: <1.5 | Zein: <1.5 |
| Low | AD: | AD: / | AD: | AD: / |
| greasiness | 0.2%~2% | | 0.2%~2% | |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A method for determining a scalp type of a user, the method comprising:
obtaining a value indicative of a humidity level of a region of the scalp of the user;
obtaining a value indicative of a sebum level in the region of the scalp of the user;
comparing a set comprising the obtained value indicative of the humidity level and the value indicative of the sebum level to reference ranges of scalp hydration levels and sebum levels, the reference ranges forming at least five scalp groups, each scalp group being associated with a humidity level range and a sebum level range, each scalp group being further associated with a corresponding category of skin and/or hair care compositions and/or skin and/or hair care treatments compatible with said humidity level range and sebum level range;
identifying, based on the comparison, a scalp type of the user among the at least five scalp groups; and
outputting the identified scalp type.

2. The method according to claim 1, wherein the at least five scalp groups are established on the basis of data extracted from scalps of at least one hundred users using at least one among:
measurements of biophysical parameter values of the scalps, the biophysical parameter values comprising a hydration level value and a sebum level value for each scalp; and
an analysis of scalp features on magnified photographs of the scalps of the at least one hundred users.

3. The method according to claim 2, wherein the value indicative of the humidity level of the region of the scalp of the user and the value indicative of the sebum level of the region of the scalp of the user are obtained using at least one among:
measurements of biophysical parameter values of the region of the scalp of the user, the biophysical parameter values comprising a hydration level value and a sebum level value of the region of the scalp of the user;
an analysis of features on magnified photographs of the region of the scalp of the user.

4. The method according to claim 3, wherein the measurement of biophysical parameter values of the scalps comprises electrical conductivity measurements or electrical resistance measurements on an upper layer of the scalp to obtain values of hydration levels and measurements of changes of translucency of absorbing strips put in contact with the scalps to obtain sebum levels.

5. The method according to claim 3 wherein the features on the magnified photographs comprise at least one among:
an intensity of light reflected by the region of the scalp;
a count of dandruff on the magnified photograph;
a count of red portions on the magnified photograph, the red portions being indicative of inflammations on the scalp; and
an amount of fluorescent light emitted from follicles of the scalp,
wherein each scalp is photographed under natural light, ultraviolet light and polarized light conditions.

6. The method according to claim 2, further comprising:
defining the at least five scalp groups (1-5) based on a visual assessment of dryness and oiliness of the scalps of the at least one hundred users, the visual assessment corresponding, for each scalp group of the at least five scalp groups, to an assessment of a similarity in the skin and/or hair care compositions and/or skin and/or hair care treatments adapted for said scalp group.

7. The method according to claim 1, wherein the reference ranges are established on the basis of data extracted from scalps of at least one hundred users, the method further comprising, for each of the at least one hundred users:
obtaining magnified photographs of the scalps of the at least one hundred users;
grouping the obtained magnified photographs according to the defined at least five scalp groups; and
for each scalp group of the at least five scalp groups, determining combinations of features found within grouped magnified photographs of scalps belonging to said scalp group.

8. The method according to claim 1, wherein the at least five scalp groups comprise:
a first scalp group (1) corresponding to a hydration level measurable as an upper skin conductivity of 0 μS to 40 μS and a Sebum level measurable as a reduction in translucency of a sebum-absorbing tape film that had been in contact with the scalp of the user between 0% and 20%;
a second scalp group (2) corresponding to a hydration level measurable as an upper skin conductivity of 0 μS to 80 μS and a Sebum level measurable as a reduction in translucency of a sebum-absorbing tape film that had been in contact with the scalp of the user between 20% and 60%;
a third scalp group (3) corresponding to a hydration level measurable as an upper skin conductivity of 0 μS to 80 μS and a Sebum level measurable as a reduction in translucency of a sebum-absorbing tape film that had been in contact with the scalp of the user between 60% and 100%;
a fourth scalp group (4) corresponding to a hydration level measurable as an upper skin conductivity of 40 μS to 80 μS and a Sebum level measurable as a reduction in translucency of a sebum-absorbing tape film that had been in contact with the scalp of the user between 0% and 40%; and
a fifth scalp group (5) corresponding to a hydration level measurable as an upper skin conductivity at or above 80 μS and a Sebum level measurable as a reduction in translucency of a sebum-absorbing tape film that had been in contact with the scalp of the user between 0% and 60%.

9. The method according to claim 8, wherein:
the first scalp group is associated with a corresponding category of skin and/or hair care compositions comprising between 10% and 18% surfactants, and have a Zein value lower than 0.75;
the second scalp group is associated with a corresponding category of skin and/or hair care compositions comprising between 12% and 20% surfactants, and have a Zein value lower than 1;
the third scalp group is associated with a corresponding category of skin and/or hair care compositions comprising between 14% and 20% surfactants, and have a Zein value lower than 2.5;
the fourth scalp group is associated with a corresponding category of skin and/or hair care compositions comprising between 10% and 18% surfactants, and have a Zein value lower than 2; and
the fifth scalp group is associated with a corresponding category of skin and/or hair care compositions comprising between 10% and 18% surfactants, and have a Zein value lower than 1.5.

10. The method according to claim 8, wherein:
the first scalp group is associated with a corresponding category of skin and/or hair care compositions comprising between 0% and 2% of an anti-dandruff agent;
the second scalp group is associated with a corresponding category of skin and/or hair care compositions comprising between 0% and 2% of an anti-dandruff agent;
the third scalp group is associated with a corresponding category of skin and/or hair care compositions comprising between 0% and 1% of an anti-dandruff agent;
the fourth scalp group is associated with a corresponding category of skin and/or hair care compositions void of anti-dandruff agent; and
the fifth scalp group is associated with a corresponding category of skin and/or hair care compositions void of anti-dandruff agent.

11. A method for recommending a skin or hair care product and/or a skin or hair care treatment to a user comprising:
determining a scalp type of the user with a method according to claim 1;
determining, based on the identified scalp type of the user, the corresponding category of skin and/or hair care compositions and/or skin and/or hair care treatments compatible with the humidity level range and sebum level range of the scalp type of the user; and
outputting the corresponding category of skin and/or hair care compositions and/or skin and/or hair care treatments.

12. The method according to claim 11, further comprising:
obtaining a magnified photograph of the region of the scalp of the user; and
upon identifying a number of red portions on the magnified photograph that is above a predetermined threshold, recommend a skin and/or hair care composition comprising a reduced proportion of surfactants adapted for sensitive scalps.

13. The method according to any one of claim 12, further comprising:
comparing a current value indicative of the humidity level of the region of the scalp of the user and a current value indicative of the sebum level of a region of the scalp of the user to previously obtained values for the region of the scalp of the user;
upon determining that the sebum level of the region of the scalp of the user increases, output a recommendation to increase the frequency of hair wash using a hair care product compatible with the scalp type of the user; and
upon determining that the sebum level of the region of the scalp of the user decreases, output a recommendation to use a hair or scalp care product comprising a lower proportion of surfactants.

14. A computer program product comprising instructions for executing a method for determining a scalp type of a user, the method comprising:
obtaining, as an input, a value indicative of a humidity level of a region of the scalp of the user;
obtaining, as an input, a value indicative of a sebum level in the region of the scalp of the user;
comparing a set comprising the obtained value indicative of the humidity level and the value indicative of the sebum level to reference ranges of scalp hydration levels and sebum levels, the reference ranges forming at least five scalp groups, each scalp group being associated with a humidity level range and a sebum level range, each scalp group being further associated with a corresponding category of skin and/or hair care compositions and/or skin and/or hair care treatments compatible with said humidity level range and sebum level range;
identifying, based on the comparison, a scalp type of the user among the at least five scalp groups; and
outputting the identified scalp type.
